(19) United States
(12) Patent Application Publication
(10) Pub. No.: US 2004/0215117 A1
(43) Pub. Date: Oct. 28, 2004

(54) IV site protector

(76) Inventor: Alison Gorman, Haddonfield, NJ (US)

Correspondence Address: DUANE MORRIS, LLP, IP DEPARTMENT, ONE LIBERTY PLACE, PHILADELPHIA, PA, 19103-7396, US

(21) Appl. No.: 10/420,294

(22) Filed: Apr. 22, 2003

(51) Int. Cl. A61F 5/00

(52) U.S. Cl. 602/3

(57) ABSTRACT

A protective covering for a site on a body part and a method of deploying the same are provided. The protective covering comprises a generally rectangular shaped strip of a waterproof material having first and second lateral edges defined between first and second end edges, a first and second surfaces, a first adhesive portion on the first surface located along the first lateral edge proximate to the first end edge, and a second adhesive portion on the first surface located along the second lateral edge proximate to the second end edge. The protective covering is wrapped a plurality of times in a partially self-overlapping manner around the body part to prevent water penetration to the site with the first surface in contact with the body part and the adhesive portions contacted to the body part to create a pair of adhesive watertight seals above and below the site.

### FIELD OF THE INVENTION

The present invention relates to limb protectors, and more particularly to waterproof coverings for intravenous catheter sites.

### BACKGROUND OF THE INVENTION

Persons with intravenous (IV) sites or peripherally inserted central catheters (PICC) must be very careful not to expose IV sites to moisture, such as during bathing. Exposure to moisture can adversely affect adhesive bandages covering the site and increase the risk of infection. The integrity of the IV site is compromised when not covered during bathing, thereby increasing the risk of infection or requiring new placement of an IV, which is uncomfortable for the patient and costly to the hospital.

Presently, as is the practice at many hospitals, the bottom portion of a plastic bag (whose primary function is to encase lab specimens) is removed to provide a tubular sheath. The patient's limb is disposed through the sheath and over the patient's IV site. Each end of the sheath is then sealed by wrapping tape around the open ends of the plastic bag. This practice suffers from several shortcomings. First, the make-shift seal often leaks, thereby compromising the IV site. Second, the configuration can be very cumbersome to employ, particularly for the patient once he or she is released from the hospital, as with a PICC. Also, the use of these biohazard bags for alternate uses is costly to the hospital.

U.S. Pat. No. 5,720,712 to Joy et al. discloses a reusable limb protector for use in protecting a limb that has a cast or bandage. The protector includes a moisture impervious material that is adjustable in size to accommodate a wide range of individuals. A watertight seal is accomplished by folding over two locking straps provided on the limb protector.

U.S. Pat. No. 5,152,282 to Elphick et al. discloses a bag having an open end for covering a bandage or IV site on, for example, an arm of a patient. The bag includes an adhesive strip along the opening of the bag and an adhesive tab on the outside surface of the bag that cooperates to create a waterproof seal for the patient's IV site or bandaged area.

Finally, U.S. Pat. No. 6,222,090 to Weston discloses a water-proof patch having an adhesive along each edge thereof for covering a catheter or other site on a patient.

A new covering for and method of covering a site on a body part that creates a watertight seal in an easily deployable manner is desired.

### SUMMARY OF THE INVENTION

A protective covering for a site on a body part of a person or animal and a method of deploying the same are provided. The protective covering comprises a generally rectangular shaped strip of a waterproof material. The strip has first and second lateral edges defined between first and second end edges, a first and second surfaces, a first adhesive portion on the first surface located along the first lateral edge proximate to the first end edge, and a second adhesive portion on the first surface located along the second lateral edge proximate to the second end edge. The protective covering is wrapped a plurality of times in a partially self-overlapping manner around the body part to prevent water penetration to the site on the body part with the first surface in contact with the body part and the adhesive portions contacted to the body part to create a pair of adhesive watertight seals above and below the site.

The above and other features will be better understood from the following detailed description of the preferred embodiments of the invention that is provided in connection with the accompanying drawings.

### BRIEF DESCRIPTION OF THE DRAWINGS

The accompanying drawings illustrate preferred embodiments of the invention, as well as other information pertinent to the disclosure, in which:

FIG. 1 is a partial, top plan view of an IV site protector;

FIG. 2 illustrates the site protector of FIG. 1 secured to cover a site on an underside of a forearm;

FIG. 3 illustrates the site protector of FIG. 1 secured to cover a site on a topside of a hand;

FIG. 4 illustrates the site protector of FIG. 1 secured to cover a site at the antecubital space of an arm;

FIG. 4A illustrates the application of the site protector of FIG. 1 to the arm of FIG. 4;

FIGS. 5A and 5B are top and bottom plan views, respectively, of an alternative embodiment of the site protector of FIG. 1;

FIG. 6A is a top plan view of an alternative embodiment of site protector;

FIG. 6B is a side elevational view of the site protector of FIG. 6A;

FIG. 7 is a top plan view of another alternative embodiment of an IV site protector;

FIG. 8 illustrates the site protector of FIGS. 5A and 5B being wrapped around a limb to cover an IV site on the topside of a hand;

FIG. 9 is a top plan view of another alternative embodiment of an IV site protector; and

FIG. 10 is a side elevational view of a reinforcing strap for use in connection with a site protector;

FIG. 10A illustrates the application of the reinforcing strap of FIG. 10 over a site protector wrapped over an IV site on a forearm.

### DETAILED DESCRIPTION

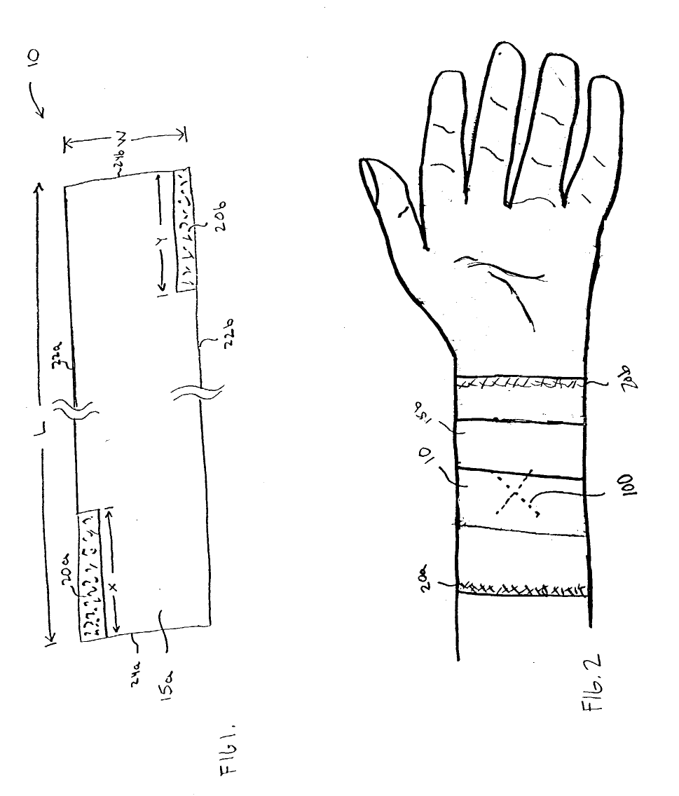

A site protector for covering intravenous catheter sites (including longer term PICC sites) is provided and illustrated in connection with FIGS. 1-9. FIG. 1 is a partial, top plan view of a protective covering 10. The protective covering is formed from a waterproof material, such as latex rubber, or possibly a silicone or a flexible polymer. A wide range of polymeric materials, consisting of polyolefins, polyamides, polyethylenes, polypropylenes, polyurethanes, polyesters, polysiloxane, acrylic, vinyl or any other suitable material, can be used for the protective covering as long as flexible, non-irritating and waterproof. Polyolefins or polyamides are preferably made of non-homogeneous blends of polyolefins or polyamides, respectively. In a preferred polymeric embodiment, polyethylenes, which are impermeable to water, serve as a component of the flexible plastic.

The protective covering 10 is generally rectangular shaped and is defined by a first lateral edge 22a, a second lateral edge 22b opposite the first lateral edge 22a, and a pair of opposite end edges 24a, 24b. By “generally rectangular,” it is meant that the protective covering may be perfectly rectangular, trapezoidal or even include rounded comers. In one embodiment, a first adhesive portion 20a is disposed along the first lateral edge 22a and proximate the first end edge 24a on first surface 15a. A second adhesive portion 20b is disposed along the first lateral edge 22b and proximate the second end edge 24b. Although it is preferred to locate the adhesive strips on opposite edges, this is not a requirement. When not in use, the adhesive portions 20a, 20b are preferably covered by removable cover strips as may be found on the typical BAND-AID ® bandage. In one embodiment, the adhesive is a self adhesive as found, for example, on some “Texas-Style catheters”, such as the HOLLISTER ® extended wear natural rubber latex self-adhesive urinary external catheter manufactured by Hollister Inc., Liberty, Ill. In one embodiment, the adhesive is a hypo-allergenic acrylic-based adhesive.

The protective covering 10 is preferably sized to wrap around a limb, such as an arm, to provide a protective, waterproof covering for an intravenous (IV) site. One of ordinary skill will recognize that limbs can certainly vary in size, and the following dimensions are provided as indicative of an exemplary protective covering for use in covering an IV site on an average person. The required size is also dependent in part on the number of loops used in wrapping the strip around a limb and the percentage of overlap between the loops.

Further, the protective covering may be sized to wrap around another body part, such as the chest, leg or abdomen of a person. Although the examples described below are used on humans, the protective covering may also be sized for use on a body part of an animal, for veterinary use, for example. Further, although an exemplary use is for covering an IV site, the covering may be used on other sites such as, for example, abrasions, bums, contusions, cuts, stitches, lesions, hives and the like. The strip may include a medication, balm, a material such as gauze coated with the same, or dry gauze or absorbent material disposed on surface 15a for treating a site on a body. As mentioned, the strip may contain various kinds of medicines, for example, a sterilizer or disinfectant, a wound astringent healing promoter, a hemostatic agent, an anti-inflammatory agent, an antihistamine, and a local anesthetic.

Specific examples of the sterilizer or disinfectant include chlorhexidine gluconate, benzalkonium chloride, chloroxylenol, acrinol, thianthol, dequalinium chloride, sulfisomidine, sulfamine, nitrofurazone, boric acid, homosulfamine, and triclocarban.

Examples of the healing promoter include zinc oxide, pyridoxine hydrochloride, tocopherol acetate, and pyridoxine dipalmitate.

Examples of the hemostatic agent include naphazoline hydrochloride, zinc sulfate, and ephedrine hydrochloride.

Examples of the anti-inflammatory agent include steroids such as prednisolone, dexamethasone, cortisone acetate and other steroids, glycyrrhetinic acid, and lysozyme chloride.

Examples of the antihistamine include chlorophenilamine maleate and diphenhydramine hydrochloride.

Examples of the local anesthetic include lidocaine, ethyl aminobenzoate, procaine hydrochloride, dibucaine hydrochloride, tetracaine hydrochloride, and diethyl aminoethyl p-butylamino-benzoate hydrochloride.

The waterproof strip is preferably provided in sufficient length such that the strip may be wrapped around the limb or body part at least three partially overlapping times over and proximate to the site (e.g., an IV site) area and at least once, and preferably twice, both above and below the site area to create the adhesive seals. The adhesive portions preferably extend along the lateral edges a sufficient length to at least completely wrap around the limb once to create an adhesive seal with the skin and to partially overlap the adjacent wrap or loop to contact the surface 15b of the site protector 10.

An exemplary site protector 10 has a length L between about 1.5′ to 5.0′, depending upon the site location and the size of the limb or body part to be covered, and a width W between about 1.5″ to 3.5″, preferably between about 2.5″ to 3.5″ and more preferably about 3.0″. The adhesive portions preferably extend along the lateral edges 22a, 22b a length sufficient to create a complete adhesive seal (i.e., ≧360°) above and below the IV site when the site protector 10 is wrapped around a limb to cover the site. The first and second adhesive portions 20a, 20b preferably extend along the lateral edges 22a, 22b between about 8.0-15.0 inches. In one embodiment, the first adhesive portion 20a extends along the first lateral edge 22a a length X of about 8.0-11.0″ and the second adhesive portion 20b extends along a second lateral edge 24b a length Y of about 10-13″. The lower range of sizes can be used on smaller limbs (e.g., on smaller persons or animals) while the higher range of sizes can be used on larger limbs (e.g., on larger persons or animals). The selected waterproof material for the cover 11 may also be stretchable to facilitate size variation as well as the creation of tight overlapping seals within the helix (described below). Each adhesive portion preferably extends along the width W of the IV site protector (i.e., along end edges 24a, 24b and onto surface 15a) about 3/16-12/16″, and preferably about ½″. It is believed that these sizes are sufficient for most uses, such as those described below. A “one size fits all” embodiment for simplicity may be about 50 inches in length with adhesive edges along the lateral edges 22a, 22b extending 10-15″ in length and ¼″ onto the width thereof.

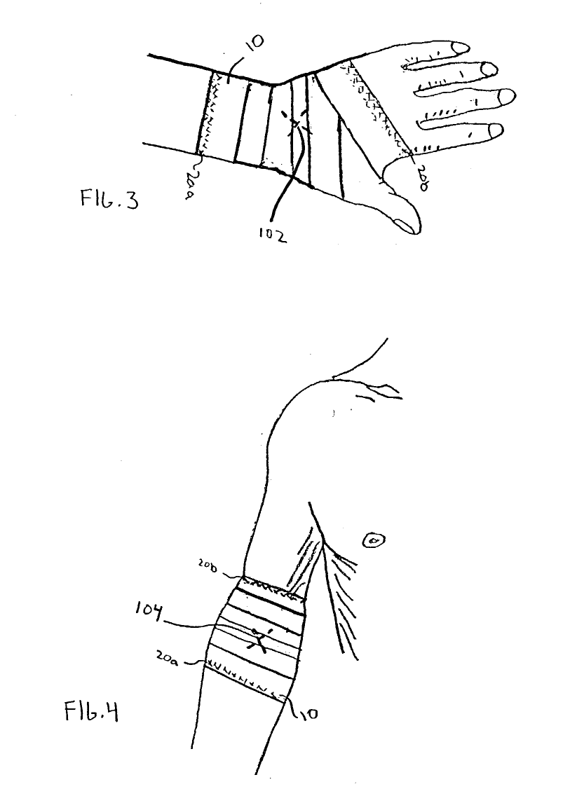

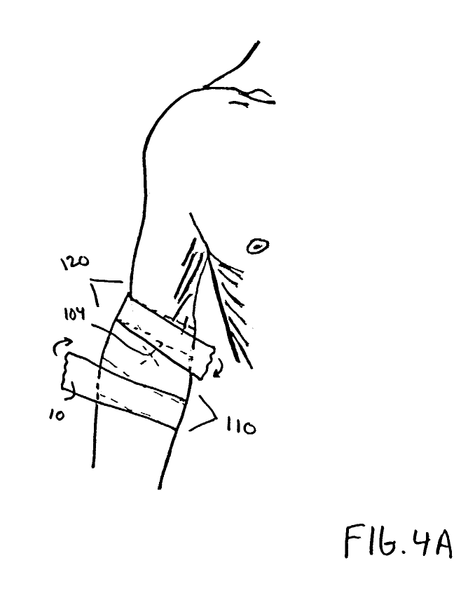

The IV site protector 10 may be used on various intravenous sites, including the top side of a hand, the forearm and at the anticubetal space of the arm, such as in connection with a peripherally inserted catheter (PICC). By way of example, FIG. 2 illustrates the site protector 10 applied to cover an IV site 100 (illustrated by a dashed “X”) on an underside of a forearm. The adhesive portions 20a, 20b, which are on surface 15a contacting the limb, are shown in cross hatch, being that they are not visible once the wrap is applied. FIG. 3 illustrates the site protector 10 applied to cover a site 102 on a topside of a hand. FIG. 4 illustrates the site protector 10 applied to cover a site 104, such as a PICC site, proximate to the antecubital space of the arm.

The application of the site protector 10 to a limb to cover a site is now described with the aid of FIG. 4A. The adhesive portions 20a, 20b are not shown in FIG. 4A for purpose of simplifying the figure. Portions of the wrap that are not visible are shown in phantom. The site protector 10 can be applied by the individual or with the aid of another person, such as a nurse. First, the covering strip over adhesive area 20a or 20b is removed. If adhesive strip 20a, which may be shorter than adhesive strip 20b, is uncovered, the site protector is first applied to the area below the IV site, i.e., the area having the smaller diameter when compared with the area above the IV site. This area is designated generally as area 110 in FIG. 4A. If adhesive strip 20b, which may be longer than adhesive strip 20a, is uncovered, the site protector is first applied to the area above the IV site. This area is designated generally as area 120 in FIG. 4A. Either side may be used if the embodiment is a “one size fits all” configuration.

After an adhesive area 20a or 20b is uncovered, the surface 15a of the site protector 10 proximate to the end edge that is proximate to the uncovered adhesive portion is pressed to the skin of the limb to begin the creation of a first adhesive seal above or below the IV site. The site protector is then wrapped a first time completely around the periphery of the limb to at least partially overlap itself such that a first watertight, adhesive seal is created either above or below the IV site 104. The adhesive preferably is disposed to contact the skin to create an adhesive seal with the skin completely around the limb and may be sized to contact the surface 15b of the strip as the strip is wrapped to completes its first loop. The site protector is then continually wrapped around the limb, in a partial overlapping manner (such as a three quarter wrap), and over the IV site 104. After the IV site is covered, the second adhesive portion (either 20a or 20b) is uncovered such that a second watertight, adhesive seal can be created below or above the IV site 104, depending upon the location of the first seal. The second adhesive seal is created in the manner just described for the first adhesive seal. The first seal is preferably created between about 2.0-3.0″ above or below the IV site, and the second seal is preferably created between about 2.0-3.0″ above or below the IV site. In essence, the protector is applied in much the same way on ACE® bandage is applied, except for the adhesive portions. Although not shown in FIGS. 4 and 4A, the IV site protector 10 may be wrapped above the elbow region as needed to sufficiently cover an antecubital site and to account for normal movement of the arm during bathing.

In this manner, a waterproof covering is provided over the IV site 104 including a plurality of overlapping seals, formed by the overlapping waterproof material, both over and above and below the IV site. The covering also preferably includes the adhesive seals above and below the IV site. The covering is easily deployed. The covering may be deployed for a single use at a low cost. Further, the same IV site protector configuration 10 can be used to cover varied IV sites, e.g., IV sites 100, 102, 104, etc, although the length of the cover strip and the adhesive portions may, but does not necessarily, vary according to size requirements.

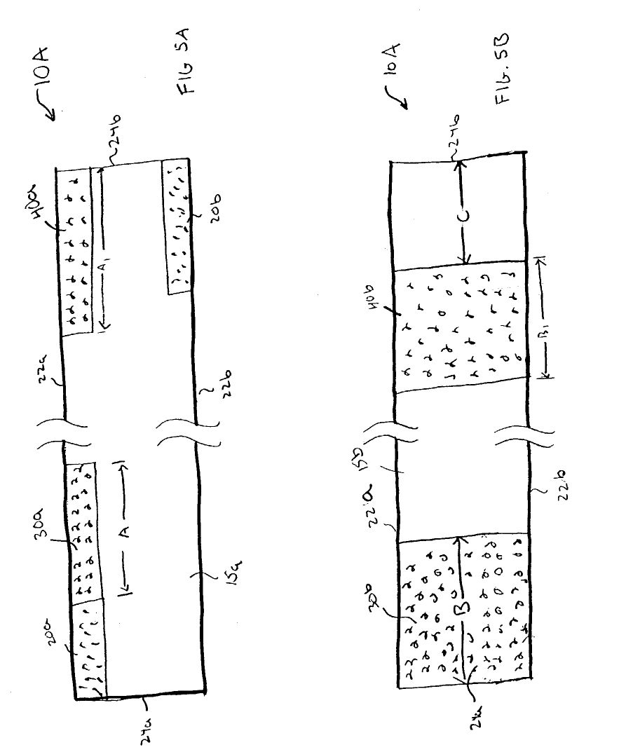

FIGS. 5A and 5B are top and bottom plan views, respectively, of an alternative embodiment of an IV site protector 10A. The IV site protector 10A is the same in all respects as the site protector 10, only the site protector 10A includes cooperable hook and loop fasteners 30a, 30b and 40a, 40b (e.g., VELCRO ® fasteners) for strengthening the first and/or second seals created by the adhesive portions 20a, 20b on the person's skin. These fastener strips may be secured to the site protector 10 by an adhesive. In one embodiment portions 30a, 40a of the cooperable hook and loop fasteners are located along the lateral edge 22a behind the adhesive portions 20a and proximate to end edge 24b, respectively, on surface 15a. Second cooperable portions 30b, 40b are provided on the opposite surface 15b for mating with portions 30a, 40a, respectively, when the site protector is wrapped around the limb. As shown, portion 40b is spaced a distance C from the end edge 24b. In this embodiment, adhesive portion 20a is used to create the first adhesive seal with the skin and adhesive portion 20b is used to create the second adhesive seal with the skin. During creation of the second seal, hook and loop fastener portion 40a overlaps the wrap segment of the helix created immediately prior to the creation of the wrap segment that forms the second adhesive seal.

In an exemplary embodiment, length C is approximately equal to length Y of second adhesive portion 20b. The cooperable hook and loop fasteners 30, 40 preferably are sized such that a complete (i.e., at least 360°) hook and loop reinforcing wrap can be created around the limb. In an exemplary embodiment, portions 30a, 30b have respective lengths A, B that are slightly longer than the length X of adhesive portion 20a (e.g., about 9.0-12.0″). Portions 40a, 40b have respective lengths A1, B1 that are slightly longer than the length Y of adhesive portion 20b (e.g., about 11.0-14.0″).

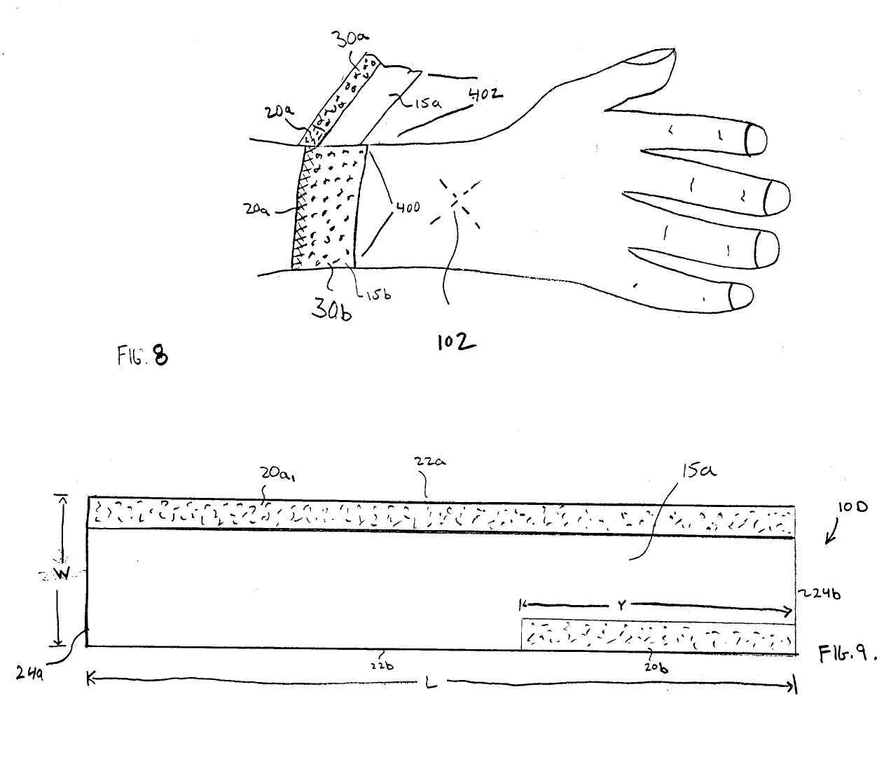

The application of the protective cover of FIGS. 5A and 5B is partially illustrated in FIG. 8. An IV site 102 is located on top side of a hand. Portion 400 of the protective cover is first applied so that adhesive portion 20a contacts the skin to begin the creation of the first adhesive seal. The adhesive portion in the portion 400 is shown in cross hatch, because it is not visible. Hook and loop fastener portion 30b is visible on surface 15b and is positioned to mate with hook and loop fastener portion 30a disposed on surface 15a when portion 402 is wrapped around the limb to complete the first adhesive seal and begin creation of the supporting hook and loop fastener seal.

Alternatively, it is envisioned that the cover may include the aforementioned hook and loop fasteners without the adhesive portions. In this embodiment, the waterproofing is provided by a combination of the tight overlapping of adjacent loops comprising the waterproof strip and the fasteners, which are employed primarily to anchor the strip at locations above and below the site to the limb.

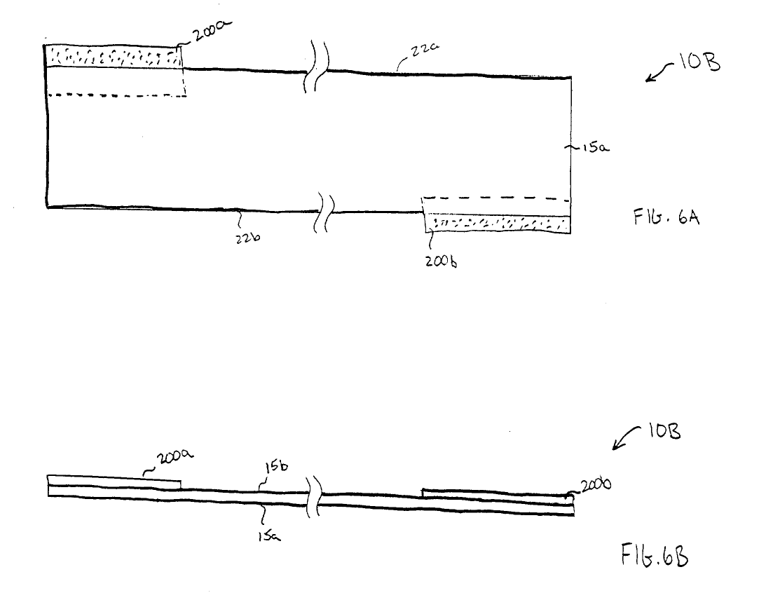

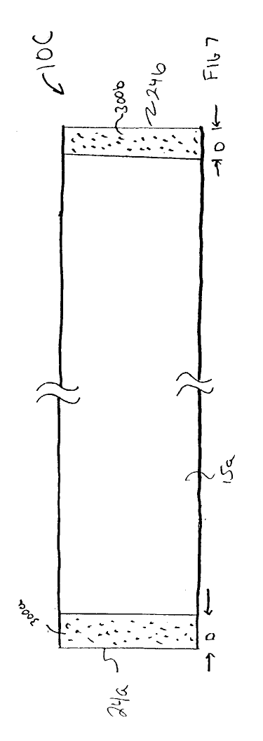

FIG. 6A is a top plan view of an alternative embodiment of an IV site protector 10B, and FIG. 6B is a side elevational view thereof. The site protector 10B is the same as site protector 10 (FIG. 1) except for the configuration of the adhesive portions. The protector 10B includes first and second adhesive strips 200a and 200b disposed along and adjacent to the first and second lateral edges 22a, 22b, respectively. The adhesive strips are secured to the surface 15b, rather than surface 15a, and extend beyond the lateral edges 22a, 22b to expose the adhesive in the direction faced by surface 15a. The portions of the adhesive strips disposed on surface 15b are shown in phantom in FIG. 6A. The exposed portions of adhesive strips 200a, 200b are sized similar to adhesive portions 20a, 20b so that a complete adhesive, watertight seal can be formed both above and below the IV site 108 when the site protector is wrapped around a limb in a helical manner and the adhesive portions 200a, 200b contact the skin.

FIG. 7 illustrates yet another embodiment of an IV site protector 10C. In this embodiment, the site protector 10C includes first and second adhesive portions 300a, 300b disposed along the end edges 24a, 24b. The adhesive portions extend onto the surface 15a preferably about ¼. In this embodiment, the adhesive portions 300a, 300b are utilized to anchor the covering strip 10B during application, not to create an adhesive seal. The watertight seals above and below the IV site and the waterproof cover over the site are created solely by the helical self-overlap of the waterproof strip.

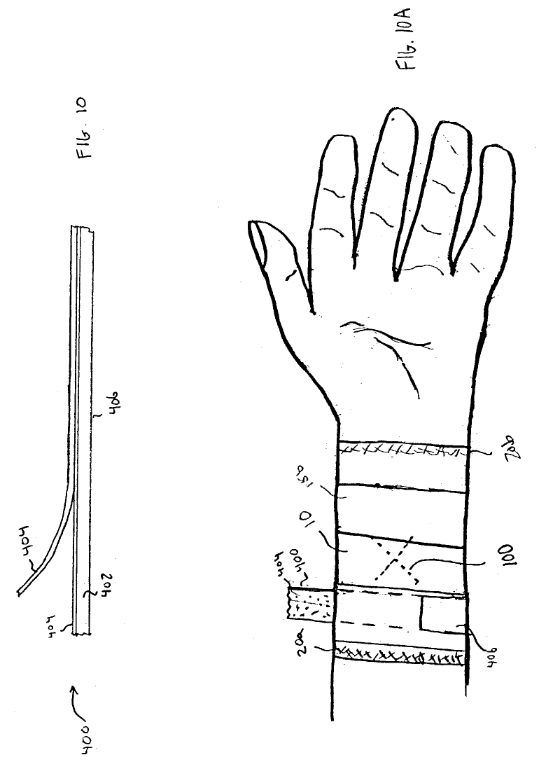

FIG. 9 illustrates an exemplary “one-size fits all” embodiment. IV site protector 10D is similar to IV site protector 10 described above, only the site protector 10D includes first adhesive portion 20a, that extends along the entire first lateral edge 22a. In one exemplary embodiment, the protector has a width W of about 3.0″ and a length L around 5.0′. Second adhesive portion 20b extends along the second lateral edge 22b a length Y of between about 10-14″, and preferably 13″. Each adhesive portion extends about ½″ onto the first surface 15a. Like site protector 10, site protector 10D is also wrapped around the limb or body part in a manner similar to an ACE ® bandage but forming a watertight seal above and below the IV site, as described above. In this embodiment, the portion of first surface 15a proximate to end edge 24a is first contacted to the patient's skin. Adhesive portion 20a, is in contact with the patient's skin when the protector 10D is first wrapped around the body part. Subsequent wraps of the overlapping helix contact the adhesive portion 20a, to the second surface 15b (not shown) of protector 10D. No adhesive comes in contact with the IV site upon wrapping, thus protecting the site and ensuring a watertight seal above and below the IV site.

FIG. 10 is a side elevational view of a reinforcing strap 400 for reinforcing a seal created above and/or below an IV site. Such straps are typically used in connection with Texas-Style catheters, as used for example with the BARD ® Disposable URO ® Sheath provided by C. R. Bard, Inc. of Covington, Ga. The strap includes a foam strip 402, an adhesive layer 404 disposed thereon and a backing cover 404 that is removed at application. The strip should be sized to have a length slightly longer than the circumference of the limb, e.g., between about 8.0-15.0 inches. It preferably has a width of about 0.5-1.0 inches. Referring to FIG. 10A, the site protector 10 is first applied to the limb as described in detail above. After the protector is applied, the cover 404 of the strap 400 is removed, and the strap 400 is wrapped around the limb at a location above (or below as selected) the IV site 100 to overlap itself. The adhesive portion contacts the protective cover 10 until the strap 400 is fully wrapped around itself, at which point the adhesive is contacted to the bottom surface 406 of the strap 400. FIG. 10A illustrates the strap partially wrapped around the forearm, with hidden portions shown in phantom. In one embodiment, a reinforcing strap 400 is applied above the IV site and a reinforcing strap 400 is applied below the IV site to reinforce both upper and lower seals, thereby providing additional protection against water entry to the IV site.

Although the invention has been described in terms of exemplary embodiments, it is not limited thereto. Rather, the appended claims should be construed broadly to include other variants and embodiments of the invention that may be made by those skilled in the art without departing from the scope and range of equivalents of the invention.

1. A protective covering for a site on body part of a person or animal, comprising:
a generally rectangular shaped strip of a waterproof material, said strip having a first and second lateral edges defined between first and second end edges, and first and second surfaces;
a first adhesive portion on said first surface located along said first lateral edge proximate to said first end edge; and
a second adhesive portion on said first surface located along said second lateral edge proximate to said second end edge,
wherein said protective covering prevents water penetration to a site on a body of a person or animal when said strip is wrapped a plurality of times in a partially self-overlapping manner around said body part to cover said site, with said first surface in contact with said body part and said adhesive portions contacted to said body part to create a pair of watertight adhesive seals with said body part above and below said site.

2. The protective cover of claim 1, wherein said first and second lateral edges are between about 18-60 inches in length and said first and second end edges are between about 1.5-3.5 inches in length.

3. The protective cover of claim 2, wherein said adhesive portions extend along said respective lateral edges between about 8-15 inches and onto said first surface between about 3/16 to 12/16 inches.

4. The protective cover of claim 3, wherein one of said adhesive portions extends along said respective lateral edge a greater distance than said other adhesive portion.

5. The protective cover of claim 1, wherein said waterproof material is selected from the group consisting of rubber latex, and a flexible plastic.

6. The protective cover of claim 1, further comprising a pair of mating hook and loop fasteners disposed on said first and second surfaces for reinforcing at least one of said watertight seals.

7. The protective covering of claim 1, wherein said first adhesive portion extends along the entire length of said first lateral edge and said second adhesive portion extends only partially along the length of said second lateral edge.

8. A method of covering a site on a body part, comprising the following steps:
providing a protective covering comprising a generally rectangular shaped strip of a waterproof material; and
helically wrapping said protective covering around said body part a plurality of times in a partially self-overlapping manner, thereby covering said site and providing a waterproof cover therefor.

9. The method of claim 8 wherein said strip has a first and second lateral edges defined between a first and second end edges, and a first and second surfaces, said strip including:
a first adhesive portion on said first surface located along said first lateral edge proximate to said first end edge; and
a second adhesive portion on said first surface located along said second lateral edge proximate to said second end edge,
said method further comprising the step of contacting said adhesive portions at least to said body part to create a pair of watertight adhesive seals above and below said site with said body part.

10. The method of claim 9, wherein said first and second lateral edges are between about 18-60 inches in length and said first and second end edges are between about 1.5-3.5 inches in length.

11. The method of claim 9, wherein said waterproof material is selected from the group consisting of rubber, latex and a flexible plastic.

12. The method of claim 9, wherein said protective covering further comprises at least one pair of mating hook and loop fasteners disposed on said first and second surfaces for reinforcing one of said watertight seals, said method further comprising the step of fastening said mating hook and loop fasteners to reinforce said one of said watertight seals

13. The method of claim 9, further comprising the steps of:
wrapping said protective covering around said body part a first time to contact said first adhesive portion to said body part to create a first watertight adhesive seal;
then, helically wrapping said protective covering around said body part a plurality of times to cover said site; and
after said helically wrapping step, wrapping said protective covering around said body part a final time to contact said second adhesive portion to said body part to create a second watertight adhesive seal.

14. The method of claim 13, wherein said first adhesive portion extends along the entire length of said first lateral edge and said second adhesive portion extends only partially along the length of said second lateral edge, said helically wrapping step including the step of contacting at least a portion of said first adhesive portion to a second surface of said protective covering.

15. The method of claim 9, wherein said site is an IV site on a limb.

16. The method of claim 8, wherein said strip has a length between about 18-60 inches and a width between about 1.5-3.5 inches.

17. The method of claim 8, wherein said strip is wrapped around said body part at least 5 times.

18. The method of claim 8, wherein said site is an IV site on a limb.

19. The method of claim 8, further comprising the step of securing at least one reinforcing strap completely around said wrapped protective covering at a location either above or below said site.

20. The method of claim 19, wherein said reinforcing strap includes an adhesive portion disposed on a foam body, sad securing step including the steps of contacting said adhesive portion of said strap to said protective cover and wrapping said strap completely around said protective cover to contact said adhesive portion to said foam body.

21. A protective covering for an intravenous (IV) site on a limb, comprising:
a generally rectangular shaped strip of a waterproof material between about 18-60 inches in length, said strip having a first and second lateral edges defined between a first and second end edges, and a first and second surfaces, said strip including a pair of adhesive portions disposed proximate to said end edges, said strip being unbroken between said first and second edges,
wherein said protective covering prevents water penetration to an IV site on a limb when said strip is wrapped a plurality of times in a partially self-overlapping manner around said limb to cover said IV site, with said first surface in contact with said limb and said adhesive portions contacted to said limb above and below said IV site.

22. The covering of claim 21, wherein said pair of adhesive portions includes:
a first adhesive portion on said first surface located along said first lateral edge proximate to said first end edge; and
a second adhesive portion on said first surface located along said second lateral edge proximate to said second end edge,
whereby a pair of watertight adhesive seals are created above and below said IV site when said protective covering is applied to said site.

23. The covering of claim 22, wherein said adhesive portions extend a length along said lateral edges between about 8-15 inches.

24. The covering of claim 22, wherein said first adhesive portion extends along the entire length of said first lateral edge and said second adhesive portion extends only partially along the length of said second lateral edge.

25. The covering of claim 21, wherein said pair of adhesive portions includes a first adhesive portion disposed adjacent to said first lateral edge and said first surface and a second adhesive portion disposed adjacent to said second lateral edge and said first surface, whereby a pair of adhesive seals are created above and below said IV site when said protective covering is applied to said site.

26. The covering of claim 25, wherein said adhesive portions extend a length along said lateral edges between about 8-15 inches.
